(19) United States
(12) Patent Application Publication
(10) Pub. No.: US 2004/0220555 A1
(43) Pub. Date: Nov. 4, 2004

(54) Endoscopic measuring tool

(75) Inventor: Masanao Abe, Saitama (JP)

Correspondence Address: GREENBLUM & BERNSTEIN, P.L.C., 1950 ROLAND CLARKE PLACE, RESTON, VA, 20191, US

(73) Assignee: PENTAX Corporation

(21) Appl. No.: 10/737,819

(22) Filed: Dec. 18, 2003

(30) Foreign Application Priority Data
Dec. 19, 2002 (JP) P.2002-369060

(51) Int. Cl. A61B 17/00; A61B 1/00

(52) U.S. Cl. 606/1; 600/104

(57) ABSTRACT

An endoscopic measuring tool used while being inserted in an endoscope, the measuring tool comprising a long portion, and a measuring portion provided at a front end portion of the long portion. The measuring portion contains TiNₓO_{y} in which 0.1≦x≦1.0, 0≦y≦1.9 and which is provided as a color former for forming a color by irradiation with laser light, in at least a part of an external surface of the measuring portion and the neighborhood of the part of the external surface. Calibrations constituted by color forming portions with the color of the color former are provided in the external surface of the measuring portion.

### BACKGROUND OF THE INVENTION

The present invention relates to an endoscopic measuring tool.

An endoscopic measuring tool is used in an endoscopic treatment for measuring a length of an affected part etc. in a coelom. As such an endoscopic measuring tool, various types of tools have been heretofore proposed (e.g. see Japanese Patent Publication No. 2001-275932).

In a related-art endoscopic measuring tool, calibrations are generally formed in a measuring portion of the measuring tool by printing using ink or by laser machining.

The method using printing has disadvantages that it is difficult to form calibrations in a curved surface, that it takes much time to dry the ink, and that the ink layer may be peeled off when in use and flow into the coelom.

On the other hand, the method using laser machining has a problem that it is difficult to recognize calibrations visually because recesses formed are set as the calibrations so that contrast between the calibrations and the other portions is not sufficiently high in comparison with the case of calibrations formed using ink. In addition, when the endoscope is pasteurized and sterilized, chemicals are apt to remain in the recesses. There is fear that deterioration will start at the recesses.

### SUMMARY OF THE INVENTION

An object of the invention is to provide an endoscopic measuring tool which has color forming portions difficult to be peeled off, eliminated or discolored as well as being excellent in visibility.

The object can be achieved by the invention described in the following (1) to (8).

(1) An endoscopic measuring tool used after inserted in an endoscope, the measuring tool including a long portion and a measuring portion provided at a front end portion of the long portion, wherein: the measuring portion contains TiNₓO_{y} in which 0.1≦x≦1.0, 0≦y≦1.9 and which is provided as a color former for forming a color by irradiation with laser light, in at least a part of an external surface of the measuring portion and the neighborhood of the part of the external surface; and calibrations constituted by color forming portions with the color of the color former are provided in the external surface of the measuring portion.

According to the invention, it is possible to obtain an endoscopic measuring tool which has color forming portions difficult to be peeled off, eliminated or discolored as well as being excellent in visibility.

(2) An endoscopic measuring tool according to (1), wherein at least the external surface of the measuring portion and the neighborhood of the external surface are mainly made of a resin material.

(3) An endoscopic measuring tool according to (1) or (2), wherein the color former is particulate.

Accordingly, the color former can be mixed (dispersed) more evenly into the external surface and its neighborhood.

(4) An endoscopic measuring tool according to (3), wherein the particulate color former has a mean particle size of not larger than 10 μm.

Accordingly, the color former can be dispersed more evenly into the external surface and its neighborhood.

(5) An endoscopic measuring tool according to any one of (1) through (4), wherein the amount of the color former contained in the portions containing the color former is in a range of from 0.01% by weight to 10% by weight.

Accordingly, the color former can be mixed sufficiently in the external surface and its neighborhood, and the color forming portions can be make clear.

(6) An endoscopic measuring tool according to any one of (1) through (5), wherein the color of the external surface before irradiation with the laser light is black or a dark color.

(7) An endoscopic measuring tool according to any one of (1) through (6), wherein the color of each of the color forming portions is white or a bright color.

(8) An endoscopic measuring tool according to (7), wherein each of the color forming portions has a whiteness (L value) of not smaller than 50%.

If the whiteness is too small, good visibility of the color forming portions cannot be obtained.

The present disclosure relates to the subject matter contained in Japanese patent application No. 2002-369060 (filed on Dec. 19, 2002), which is expressly incorporated herein by reference in their entireties.

### BRIEF DESCRIPTION OF THE DRAWINGS

FIG. 1 is a partly sectional side view showing an embodiment of an endoscopic measuring tool according to the invention.

FIG. 2 is a partly longitudinal sectional view showing a front end portion of the endoscopic measuring tool depicted in FIG. 1 (in a state in which a measuring portion of the measuring tool is opened).

FIG. 3 is a side view showing the front end portion of the endoscopic measuring tool depicted in FIG. 1 (in a state in which the measuring portion of the measuring tool is closed)

FIG. 4 is a view for explaining the usage of the endoscopic measuring tool depicted in FIG. 1.

FIG. 5 is a view for explaining the usage of the endoscopic measuring tool depicted in FIG. 1.

FIG. 6 is a view for explaining the usage of the endoscopic measuring tool depicted in FIG. 1.

### DETAILED DESCRIPTION OF PREFERRED EMBODIMENTS

An endoscopic measuring tool according to the invention will be described below in detail with reference to the accompanying drawings and in connection with a preferred embodiment.

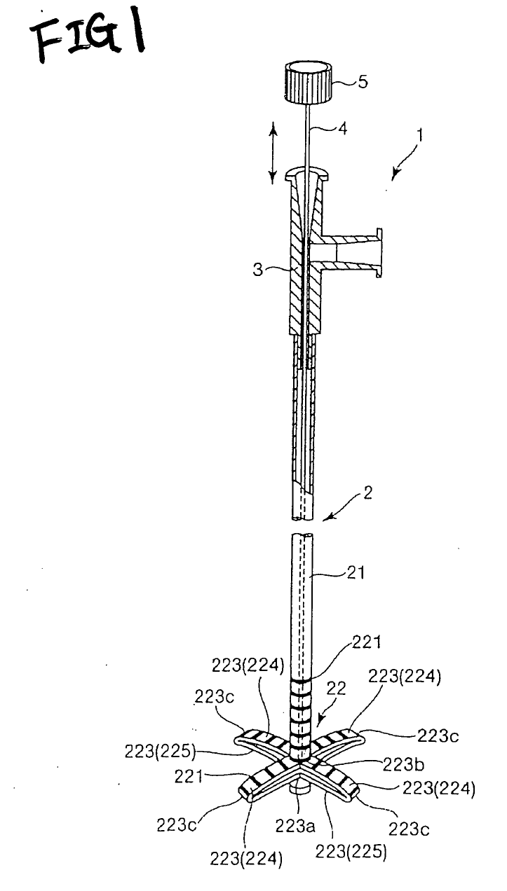

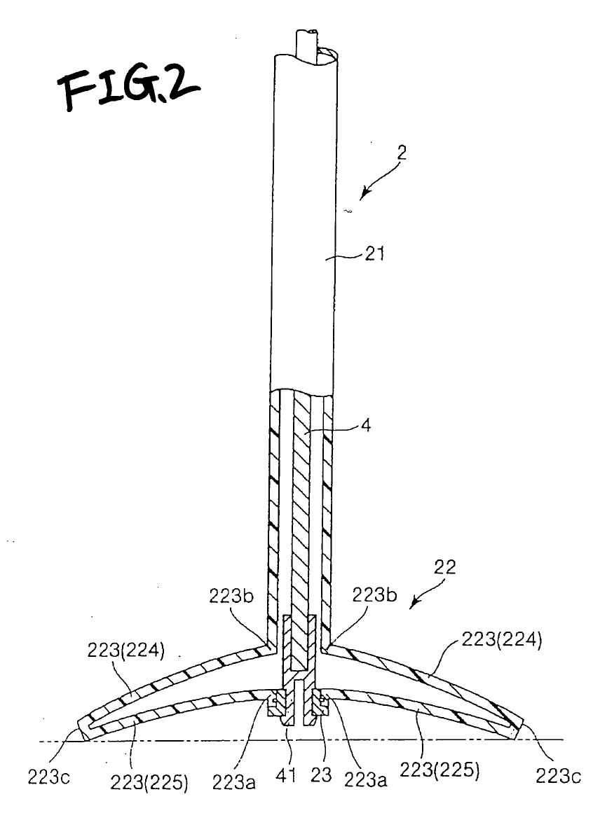

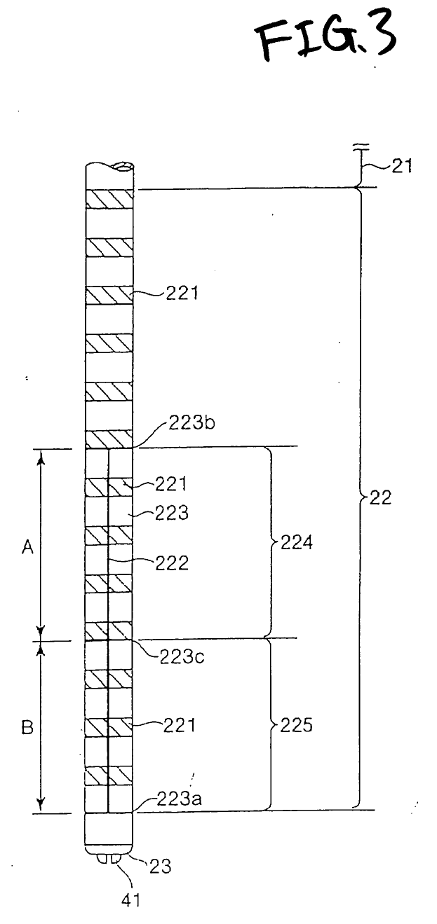

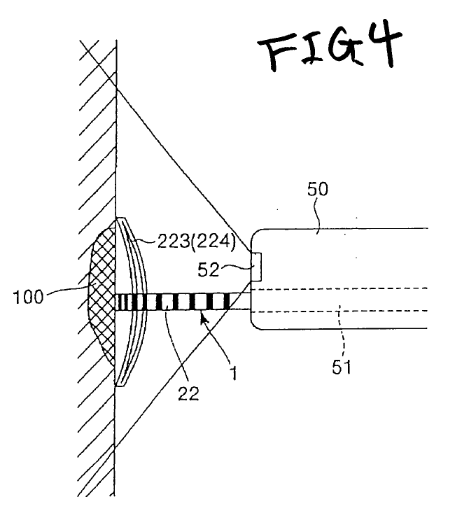

FIG. 1 is a partly sectional perspective view showing an embodiment of an endoscopic measuring tool according to the invention. FIG. 2 is a partly longitudinal sectional view of a front end portion of the endoscopic measuring tool depicted in FIG. 1 (in a state in which a measuring portion of the measuring tool is opened). FIG. 3 is a side view of the front end portion of the endoscopic measuring tool depicted in FIG. 1 (in a state in which the measuring portion of the measuring tool is closed). FIGS. 4 to 6 are views for explaining how to use the endoscopic measuring tool depicted in FIG. 1. Incidentally, in FIGS. 1 to 3, upper and lower sides will be hereinafter referred to as “base end” and “front end” respectively.

The endoscopic measuring tool (endoscopic measure) 1 depicted in FIG. 1 is used after inserted in an endoscope. The measuring tool 1 includes a sheath 2 inserted in a treatment tool insertion channel of the endoscope, a hub 3 provided at a base end of the sheath 2, an operating wire 4 inserted in the hub 3 and the sheath 2, and a grip portion 5 provided at a base end of the operating wire 4.

The sheath 2 is made of a long flexible member (tube) and curved in accordance with a curved state of the endoscope when the sheath 2 is inserted in the endoscope.

The sheath 2 has an outer diameter preferably selected to be in a range of from about 1.5 mm to about 3.0 mm, more preferably in a range of from about 1.7 mm to about 2.5 mm, and an inner diameter preferably selected to be in a range of from about 1.4 mm to about 2.9 mm.

There is no particular limitation to the material for forming the sheath 2. Examples of the material include: polyolefin such as polyethylene, polypropylene, ethylene-propylene copolymer, and ethylene-vinyl acetate copolymer (EVA); cyclic polyolefin; modified polyolefin; polyvinyl chloride; polyvinylidene chloride; polystyrene; polyamide; polyimide; polyamide-imide; polycarbonate; poly-(4-methylpentene-1); ionomer; acrylic resin; polymethyl methacrylate; acrylonitrile-butadiene-styrenecopolymer (ABS resin); acrylonitrile-styrene copolymer (AS resin); butadiene-styrene copolymer; polyoxymethylene; polyvinyl alcohol (PVA); ethylene-vinyl alcohol copolymer (EVOH); polyester such as polyethylene terephthalate (PET), polybutylene terephthalate (PBT), and polycyclohexane terephthalate (PCT); polyether; polyether-ketone (PEK); polyether-ether-ketone (PEEK); polyether-imide; polyacetal (POM); polyphenylene oxide; modified polyphenylene oxide; polysulfone; polyether-sulfone; polyphenylene sulfide; polyarylate; aromatic polyester (liquid crystal polymer); polytetrafluoroethylene (PTFE); tetrafluoroethylene-perfluoroalkoxy vinyl ether copolymer (PFA); tetrafluoroethylene-hexafluoropropylene copolymer (FEP); polyvinylidene fluoride; other fluororesins; various kinds of thermoplastic elastomers such as styrene, polyolefin, polyvinyl chloride, polyurethane, polyester, polyamide, polybutadiene, trans-polyisoprene, fluoro rubber, and chlorinated polyethylene; butadiene-based rubber such as natural rubber (NR), isoprene rubber (IR), butadiene rubber (BR, 1,2-BR), and styrene butadiene rubber (SBR); diene-based special rubber such as chloroprene rubber (CR), and butadiene-acrylonitrile rubber (NBR); olefin-based rubber such as butyl rubber (IIR), ethylene-propylene rubber (EPM, EPDM), acrylicrubber (ACM, ANM), andhalogenatedbutyl rubber (X-IIR); urethane-based rubber such as urethane rubber (AU, EU); ether-based rubber such as hydrin rubber (CO, ECO, GCO, EGCO); polysulfide-based rubber such as polysulfide rubber (T); various kinds of rubber such as silicone rubber (Q), fluoro rubber (FKM, FZ), and chlorinated polyethylene (CM); and copolymers, blends or polymer alloys mainly based on these various materials. One kind of material selected from these materials may be used or two or more kinds of materials selected from these materials may be used in combination.

Among these materials, polytetrafluoroethylene (PTFE), tetrafluoroethylene-perfluoroalkoxy vinyl ether copolymer (PFA), tetrafluoroethylene-hexafluoropropylene copolymer (FEP), etc. may be especially preferably used as the material for forming the sheath 2. Since these materials are excellent in chemical resistance, the durability of the sheath 2 can be improved against a cleaning, pasteurization and sterilization process repetitively applied to the endoscopic measuring tool 1.

The sheath 2 has a body portion (long portion) 21, and a measuring portion 22 provided in a front end portion of the body portion 21 and having calibrations 221 formed along a lengthwise direction of an external surface of the measuring portion 22. The body portion 21 and the measuring portion 22 are formed so as to be integrated with each other. The calibrations 221 can be used for measuring the dimensions of a predetermined part (affected part) in a coelom endoscopically (through an endoscopic treatment).

As shown in FIG. 3, a plurality of notches (grooves) 222 (four in this embodiment) are formed on a front end side of the measuring portion 22 and approximately in parallel with the lengthwise direction of the sheath 2. The notches 222 are approximately equal in length to one another and provided at regular intervals of approximately 90° in the circumferential direction of the sheath 2. Accordingly, the measuring portion 22 is divided into a plurality of strip pieces 223 (four) by the notches 222.

Flexion lines 223a and 223b are formed at front and base ends of each strip piece 223 respectively so that the strip piece 223 can be folded with its outer side in along the flexion lines 223a and 223b. A flexion line 223c is formed in an intermediate portion between the flexion lines 223a and 223b of each strip piece 223 so that the strip piece 223 can be folded with its outer side out along the flexion line 223c. Accordingly, each strip piece 223 is provided so that the lengthwise center portion of the strip piece 223 can be deformed to become distant from the central axis of the sheath 2, that is, can be protruded outward from the outer circumferential surface of the sheath 2.

A length (length A in FIG. 3) between the flexion lines 223c and 223b (of each base end side strip piece 224) is selected to be larger than a length (length B in FIG. 3) between the flexion lines 223c and 223a (of a corresponding front end side strip piece 225).

The operating wire 4 which is flexible (elastic) is inserted in the sheath 2 so that the operating wire 4 can move back and forth in the lengthwise direction (axial direction) of the sheath 2. As shown in FIG. 2, an engagement tip 41 attached to a front end of the operating wire 4 is engaged with an engagement member 23 attached to the front end of the sheath 2, so that the front end of the operating wire 4 is fixed to the front end of the sheath 2.

The hub 3 is anchored (fixed) to the base end of the sheath 2. The grip portion 5 attached to a base end of the operating wire 4 can be engaged/disengaged with/from the hub 3.

In the condition that the grip portion 5 is engaged with the hub 3, that is, in the condition that the operating wire 4 is most deeply forced into the sheath 2, a diameter formed by the respective strip pieces 223 is reduced so that the outer diameter of the sheath 2 as a whole is kept approximately uniform. On the other hand, when the grip portion 5 is disengaged from the hub 3 and the operating wire 4 is pulled toward the upper (base end) side relative to the sheath 2, the respective strip pieces 223 protrude outward from the outer circumferential surface of the sheath 2. As described above, the length A of the base end side strip piece 224 is larger than the length B of the front end side strip piece 225. Accordingly, when the operating wire 4 is pulled toward the upper side relative to the sheath 2, the respective strip pieces 223 protrude outward from the outer circumferential surface of the sheath 2 in the condition that the strip pieces 223 are warped forward with respect to a direction approximately perpendicular to the central axis of the sheath 2, as shown in FIG. 2.

In this embodiment, the length of the base end side strip piece 224 and the length of the front end side strip piece 225 are set so that the protrusion end (the flexion line 223c and its neighborhood) of each of the strip pieces 223 is located in front of or on approximately the same plane with the front end of the sheath 2 when the operating wire 4 is pulled to the utmost toward the base end side relative to the sheath 2.

Although the embodiment has been described on the case where the measuring portion 22 and the body portion 21 are formed so as to be integrated with each other, the invention may be also applied to the case where the measuring portion 22 and the body portion 21 are provided separately and connected (joined) to each other.

The sheath 2 contains a color former. The calibrations (visible markers) 221 each constituted by a color forming portion with the color of the color former are provided in a predetermined area of the external surface of the sheath 2.

In the embodiment, as shown in FIG. 1, the calibrations 221 each having a width of 1 mm to 10 mm are provided at intervals of 1 cm (pitch) along the lengthwise direction of the sheath 2. Incidentally, the interval between every two calibrations 221 may be set desirably.

In the invention, TiNₓO_{y} (in which 0.1≦x<1.0, 0≦y≦1.9) is used as the color former. If x in TiNₓO_{y} is smaller than 0.1, there is the possibility that color may be not formed well. TiNₓO_{y} can hardly contain N having x larger than 1.0. On the other hand, if y in TiNₓO_{y} is larger than 1.9, it is impossible to obtain a good color tone.

The ratio O/N by weight in TiNₓO_{y} is preferably in a range of from about 0.2 to about 8, more preferably in a range of from about 0.3 to about 7.

The color former may be formed into any shape such as particles, granules, pellets or flakes. Particularly, the color former may be preferably particulate. In this manner, the color former can be mixed (dispersed) more evenly into the sheath 2 (the material for forming the sheath 2).

When a particulate color former is used, the mean particle size of the color former is not particularly limited. For example, the mean particle size is selected to be preferably not larger than 10 μm, more preferably not larger than 1 μm, further preferably in a range of from about 0.1 μm to about 1 μm. In this manner, the color former can be more evenly dispersed into the sheath 2.

The amount of the color former contained in the sheath 2 depends on the composition and characteristic (particularly, color tone, etc.) of the resin material. In order to form a color sufficiently well, the amount of the color former contained in the sheath 2 as a whole is selected to be preferably in a range of from about 0.01% by weight to about 10% by weight, more preferably in a range of from about 0.01% by weight to about 1% by weight. If the amount of the color former is too small, the formed color may be obscure because whiteness of each of the color forming portions (calibrations 221) is low. On the other hand, if the amount of the color former is larger than the aforementioned upper limit, it may be difficult to mix the color former into the sheath 2 (the material for forming the sheath 2) as well as increase in effect cannot be expected any more. In the invention, the color tone or intensity of the formed color after irradiation with laser light can be adjusted according to the amount of the color former.

Although the color former in the sheath 2 is preferably dispersed evenly, the color former maybe localized, for example, on the external surface side of the sheath 2 (i.e., in the external surface of the sheath 2 and its neighborhood), or only in the measuring portion 22, particularly only in the areas where the color forming portions (calibrations 221) are provided and the neighborhoods of the areas.

The composition of other components (i.e., the mixture ratio of components contained in the sheath 2) may be even over the whole of the sheath 2 or uneven according to respective regions of the sheath 2. For example, the components may have such concentration gradients (gradient components) that the mixture ratio of components varies gradually in the direction of the thickness of the sheath 2.

Incidentally, a colorant may be added (mixed) into the material for forming the sheath 2, if necessary.

Examples of the colorant include: various kinds of dyes such as nitro so dye, nitro dye, azo dye, stilbene azo dye, ketimine dye, triphenylmethane dye, xanthene dye, acridine dye, quinoline dye, methine dye, thiazole dye, indamine dye, azine dye, oxazine dye, thiazine dye, sulfur dye, aminoketone dye, anthraquinone dye, indigoid dye, quinophthalone dye, and anthrapyridone dye; various kinds of organic pigments such as azo pigment, disazo pigment, phthalocyanine pigment, quinacridone pigment, perylene pigment, perinone pigment, dioxazine pigment, anthraquinone pigment, and isoindolinone (isooxindole) pigment; and inorganic pigments such as lead sulfate, titan yellow, iron oxide pigment, ultramarine blue, cobalt blue, chrome oxide green, spinel green, zinc yellow, chrome vermilion, chrome yellow, chrome green, cadmium yellow, cadmium red, carbon powder, zinc oxide, and titanium oxide. One kind selected from these may be used or two or more kinds selected from these may be used in combination.

When the colorant is added into the material for forming the sheath 2, the amount of the added colorant (the colorant content) is not particularly limited. The amount of the added colorant is selected to be preferably in a range of from 0.001 parts by weight to 1 part by weight, more preferably in a range of from 0.01 parts by weight to 0.1 parts by weight, with respect to 100 parts by weight of the resin material. If the amount of the added colorant is too small, the effect expected to be generated by addition of the colorant may not be exerted depending on the kind of the colorant, etc. On the other hand, if the amount of the added colorant is too large, the sheath 2 may be etched by irradiation with laser light depending on the kind of the resin material, etc.

Further, another additive may be added (mixed) into the material for forming the sheath 2, if necessary.

Examples of the additive include inorganic filler, lubricant, plasticizer, various kinds of stabilizers (such as anti-oxidant, photostabilizer, antistatic, and antiblock), releasant, fire retardant, coupler, and X-ray contrast media.

Examples of the inorganic filler include: silicon compounds such as silicon dioxide, mica, kaolin, blast furnace slag, silica sand, diatomaceous earth, and talc; calcium carbonate; alumina; glass fiber; wollastonite; glass flakes; milled fiber; hardened zinc whisker; and potassium titanate whisker.

Examples of the lubricant include: stearic acid and behenic acid, and esters or salts thereof; waxes such as carnauba wax, and polyethylene wax; and various kinds of surface active agents.

Examples of the plasticizer include phthalic ester, phosphoric ester, and sebacic ester.

Visible markers (color forming portions) each having another shape may be provided in place of the calibrations 221 or in combination with the calibrations 221. Any form such as a polka-dot pattern, a lattice pattern, a net pattern, a numeric pattern, a character pattern or a symbolic pattern may be used as the other shape (pattern) of each of the visible markers if the form can be recognized visually. Two or more different kinds of these patterns may be used in combination.

Next, a method for forming each of the calibrations (color forming portions) 221 will be described.

When a predetermined area in the external surface of the color former-containing sheath 2 is irradiated with laser light, the energy of the laser light makes the color former form a color and the irradiated portion rises by 1 to 500 μm. In this manner, the calibrations 221 are formed.

Examples of the laser light applied include carbon dioxide gas laser light, He—Ne laser light, ruby laser light, semiconductor laser light, argon laser light, excimer laser light, and YAG laser light.

Especially, YAG laser light is preferred for the following reason. YAG laser light has a wavelength of 1.06 μm, so that the energy of the YAG laser light is not substantially absorbed to the resin material which is a main component of the sheath 2. Hence, the resin material is hardly burned or vaporized, so that the sheath 2 is hardly etched with the YAG laser light.

A laser irradiation apparatus is not particularly limited. Any known apparatus such as a scanning type apparatus, a dot type apparatus or a mask type apparatus may be used as the laser irradiation apparatus.

Either continuous oscillation or pulse oscillation may be used as the mode of oscillation of the laser light.

Before such laser light is applied, the color of the external surface of the sheath 2 exhibits black or a dark color due to TiNₓO_{y} (black). When the external surface of the sheath 2 is irradiated with the laser light, TiNₓO_{y} (black) changes to TiO₂ (white) because of a high temperature oxidation reaction. Accordingly, the color of the calibrations 221 (color forming portions) exhibits white or a bright color. When TiNₓO_{y} is used as the color former as described above, a very high brightness difference (contrast) can be obtained between the calibrations 221 (color forming portions) and the other portions in the measuring portion 22.

The whiteness (L value) of each calibration 221 (color forming portion) is selected to be preferably not smaller than 50%, more preferably not smaller than 60%. If the whiteness is too small, it is impossible to obtain good visibility of the calibration 221.

When the aforementioned colorant is added (mixed) into the material for forming the sheath 2, the contrast between the calibrations 221 and the other portions can be enhanced more greatly in (the measuring portion 22 of) the sheath 2.

As described above, the calibrations 221 are formed in such a manner that the color former irradiated with laser light forms a color. Accordingly, the calibrations 221 per se are hardly peeled off, eliminated or discolored. Further, since the calibrations 221 are not formed as recesses in the external surface of (the measuring portion 22 of) the sheath 2, chemicals hardly remain in the portions of the calibrations 221 when the endoscopic measuring tool 1 is disinfected or sterilized. Accordingly, deterioration of (the measuring portion 22 of) the sheath 2 can be prevented or suppressed.

According to the invention, such an ink drying process as required at the time of forming the calibrations 221 on the external surface of the sheath 2 by printing is made unnecessary. There is an advantage that the calibrations 221 can be formed in a short time.

TiO₂ forming the calibrations 221 has the property of being incapable of transmitting X rays, so that TiO₂ can improve the handling property of the endoscopic measuring tool 1 under roentgenoscopy.

Although this embodiment has been described on the case where the sheath 2 has one single layer, the invention may be also applied to the case where part or all of the sheath 2 in the lengthwise direction is made of a laminate of a plurality of layers. In this case, at least the outermost layer of the laminate can be formed to have the same configuration as that of the sheath 2 according to this embodiment.

Next, an example of the usage (effect) of the endoscopic measuring tool 1 will be described.

Before the endoscopic measuring tool 1 is inserted into the treatment tool insertion channel of the endoscope, the grip portion 5 is engaged with the hub 3 and a diameter formed by the strip pieces 223 is reduced. Accordingly, the endoscopic measuring tool 1 can be inserted into the treatment tool insertion channel 51 of the endoscope 50 easily.

When the dimensions of a predetermined part (affected part, etc.) in a coelom are measured, the grip portion 5, in the condition in which the respective strip pieces 223 protrude from a front end of the endoscope 50, is detached from the hub 3 and pulled toward the upper (base end) side relative to the sheath 2. In this manner, as shown in FIG. 1, the strip pieces 223 protrude outward from the outer circumferential surface of the sheath 2 so as to be shaped like a cross as a whole.

FIG. 4 shows a state in which the respective strip pieces 223 protruding from the treatment tool insertion channel 51 of the endoscope 50 are pressed against the affected part 100 in the coelom. In this state, respective protrusion ends (the flexion lines 223c and their neighborhoods) of the strip pieces 223 are located in front of or on approximately the same plane with the front end of the sheath 2. Accordingly, the respective protrusion ends of the strip pieces 223c an be brought into contact with a mucomembranous surface of the affected part 100 so as not to float up from the affected part 100.

Accordingly, the dimensions of the affected part 100 can be measured accurately on the basis of observation through an observation window 52 of the endoscope 50, because there is no parallax error produced between the affected part 100 and the calibrations 221 formed in the base end side strip pieces 224 (or because parallax error is very small even in the case where the parallax error is produced). Incidentally, when the respective protrusion ends of the strip pieces 223 are located in front of the front end of the sheath 2, the same effect can be obtained even in the case where the affected part 100 exists on an inclined surface.

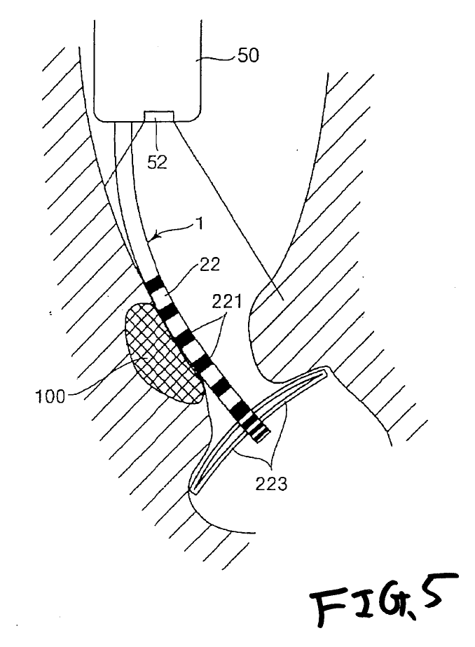

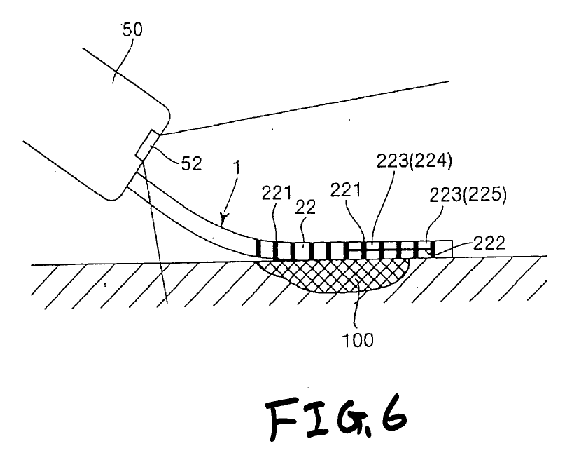

As shown in FIG. 5, the length of the affected part 100 can be measured when the base end side of the measuring portion 22 is located along the affected part 100. As shown in FIG. 6, the length of the affected part 100 can be measured by use of the whole of the measuring portion 22 in the condition that the diameter formed by the strip pieces 223 is reduced.

Although the endoscopic measuring tool according to the invention has been described above in connection with the embodiment illustrated in the drawings, the invention is not limited thereto.

At least the external surface of the measuring portion and its neighborhood may be made of a material which contains a silicon compound such as alkoxysilane or hydrolyzed alkoxysilane, and a color former added to the silicon compound.

### EXAMPLES

Specific examples will be described below.

### Example 1

A tube (with a mean thickness of 0.4 mm and a total length of 2000 mm) was produced from polytetrafluoroethylene (made by DAIKIN INDUSTRIES, LTD.) containing TiN_{0.3}O_{1.3} (color former)

Incidentally, particles each having a mean particle size of 0.4 μm were used as particles of TiN_{0.3}O_{1.3}. The amount of TiN_{0.3}O_{1.3} contained in the tube was set at 0.1% by weight.

Then, four grooves each having a length of 5 mm were formed in the front end portion of the tube so as to be arranged at intervals of 90° in a circumferential direction of the tube.

Then, the external surface of the tube was irradiated with YAG laser light so that calibrations were formed in the external surface of the tube. In this manner, a measuring portion was formed, so that a sheath shown in FIG. 1 was obtained. The sheath was used for manufacturing an endoscopic measuring tool.

### Examples 2 to 6

In each of Examples 2 to 6, an endoscopic measuring tool was manufactured in the same manner as in Example 1 except that the condition of the color former and the kind of the material for forming the sheath were changed as shown in Table 1.

### Comparative Example 1

A tube (with a mean thickness of 0.4 mm and a total length of 2000 mm) was produced from polytetrafluoroethylene (made by DAIKIN INDUSTRIES, LTD.) containing no color former.

Then, four grooves each having a length of 5 mm were formed in the front end portion of the tube so as to be arranged at intervals of 90° in a circumferential direction of the tube.

Then, the external surface of the tube was printed with fluorine-based paint (ink) to thereby form calibrations. Thus, a measuring portion was formed, so that a sheath was obtained. The sheath was used for manufacturing an endoscopic measuring tool.

### Comparative Example 2

A tube (with a mean thickness of 0.4 mm and a total length of 2000 mm) was produced from polytetrafluoroethylene (made by DAIKIN INDUSTRIES, LTD.) containing no color former.

Then, four grooves each having a length of 5 mm were formed in the front end portion of the tube so as to be arranged at intervals of 90° in a circumferential direction of the tube.

Then, the external surface of the tube was laser-machined with YAG laser light to thereby form calibrations as recesses. Thus, a measuring portion was formed, so that a sheath was obtained. The sheath was used for manufacturing an endoscopic measuring tool.

### Evaluation

The following evaluation tests I to III were carried out on the endoscopic measuring tool manufactured in each of Examples 1 to 6 and Comparative Examples 1 and 2.

Evaluation Test I (Whiteness Evaluation)

The whiteness (L value) of each calibration portion in the endoscopic measuring tool manufactured in each of Examples 1 to 6 and Comparative Examples 1 and 2 was measured with a whiteness checker (NW-1 made by NIPPON DENSHOKU INDUSTRIES CO., LTD.) and evaluated in accordance with the following two-stage criterion.

∘: not smaller 50%

X: smaller than 50%

Evaluation Test II (Visibility Evaluation)

Each calibration in the endoscopic measuring tool manufactured in each of Examples 1 to 6 and Comparative Examples 1 and 2 was checked by eye observation, and the visibility of the calibration was evaluated in accordance with the following four-stage criterion.

⊚: very good

∘: good

Δ: a little poor

X: poor

Evaluation Test III (Durability Evaluation)

The endoscopic measuring tool manufactured in each of Examples 1 to 6 and Comparative Examples 1 and 2 was subjected to a process for cleaning the endoscopic measuring tool with an enzyme-based detergent and then sterilizing the endoscopic measuring tool by high-pressure steam (condition: 132° C. per 5 min). The process was repeated by 50 cycles. After completion of the 50 cycles, the calibrations and their neighborhoods were checked by eye observation in order to make evaluation in accordance with the following four-stage criterion.

⊚: Calibrations were kept clear and there was no deterioration observed in the measuring portion.

∘: Calibrations were a little unclear but there was no deterioration observed in the measuring portion.

Δ: Calibrations were unclear and the roughness of the measuring portion was observed in the calibrations and their neighborhoods.

X: Calibrations were not recognized at all and the roughness of the measuring portion was conspicuous in the calibrations and their neighborhoods.

Results of the evaluation tests I to III are shown in Table 1, together with the condition of the color former and the kind of the material for forming the sheath.

TABLE 1

| | Measuring Portion (Sheath) | | | | | | | |
|---|---|---|---|---|---|---|---|---|
| | TiNₓO_{y} | | | | | | | |
| | | | Mean | | | | | |
| | | | Particle | Content | Resin | Evaluation Test | | |
| | x | y | Size (μm) | [wt %] | Material | I | II | III |
| Example 1 | 0.3 | 1.3 | 0.4 | 0.1 | PTFE | ◯ | ⊚ | ⊚ |
| Example 2 | 0.3 | 1.3 | 0.4 | 0.5 | PTFE | ◯ | ⊚ | ⊚ |
| Example 3 | 0.3 | 1.3 | 0.4 | 1.0 | PTFE | ◯ | ⊚ | ⊚ |
| Example 4 | 0.3 | 1.3 | 0.4 | 3.0 | PTFE | ◯ | ⊚ | ⊚ |
| Example 5 | 0.3 | 1.3 | 0.4 | 5.0 | PTFE | ◯ | ⊚ | ⊚ |
| Example 6 | 0.3 | 1.3 | 0.4 | 10.0 | PTFE | ◯ | ⊚ | ⊚ |

| | | | | | |
|---|---|---|---|---|---|
| Comparative | None | PTFE | ◯ | ⊚ | X |
| Example 1 | | | | | |
| Comparative | None | PTFE | X | X | X |
| Example 2 | | | | | |

PTFE: polytetrafluoroethylene

As shown in Table 1, in the endoscopic measuring tool manufactured in each of Examples 1 to 6, the whiteness (L value) of each calibration was high, the visibility of the calibration was very good, and the calibration was kept clear even after completion of the 50 cycles in the evaluation test III.

On the contrary, in the endoscopic measuring tool manufactured in Comparative Example 1, the whiteness (L value) of each calibration was high and the visibility of the calibration was very good immediately after manufacturing, but the durability of the calibration was poor and the calibration was peeled off after completion of the 50 cycles in the evaluation test III.

In the endoscopic measuring tool manufactured in Comparative Example 2, each calibration was low in whiteness (L value) and poor in both visibility and durability, and the roughness was conspicuous in the outer layer in the calibration and its neighborhood after completion of the 50 cycles in the evaluation test III.

As described above, in accordance with the invention, a very high brightness difference can be obtained between the color forming portions and the other portions, so that excellent visibility can be obtained. The color forming portions are hardly peeled off, eliminated or discolored. The external surface can be restrained from being deteriorated in the color forming portions and their neighborhoods because of the formation of the color forming portions.

What is claimed is:

1. An endoscopic measuring tool used while being inserted in an endoscope, the measuring tool comprising a long portion, and a measuring portion provided at a front end portion of the long portion, wherein:
the measuring portion contains TiNₓO_{y} in which 0.1≦x≦1.0, 0≦y≦1.9 and which is provided as a color former for forming a color by irradiation with laser light, in at least a part of an external surface of the measuring portion and the neighborhood of the part of the external surface; and
calibrations constituted by color forming portions with the color of the color former are provided in the external surface of the measuring portion.

2. The endoscopic measuring tool according to claim 1, wherein at least the external surface of the measuring portion and the neighborhood of the external surface are mainly made of a resin material.

3. The endoscopic measuring tool according to claim 1, wherein the color former is particulate.

4. The endoscopic measuring tool according to claim 3, wherein the particulate color former has a mean particle size of not larger than 10 μm.

5. The endoscopic measuring tool according to claim 1, wherein the amount of the color former contained in the portions containing the color former is in a range of from 0.01% by weight to 10% by weight.

6. The endoscopic measuring tool according to claim 1, wherein the color of the external surface before irradiation with the laser light is black or a dark color.

7. The endoscopic measuring tool according to claim 1, wherein the color of each of the color forming portions is white or a bright color.

8. The endoscopic measuring tool according to claim 7, wherein each of the color forming portions has a whiteness (L value) of not smaller than 50%.
